(19) United States
(12) Patent Application Publication
(10) Pub. No.: US 2013/0035548 A1
(43) Pub. Date: Feb. 7, 2013

(54) FIBER OPTIC INTUBATING DEVICE

(71) Applicant: Stefan Alexandrov Ianchulev, Marblehead, MA (US)

(72) Inventor: Stefan Alexandrov Ianchulev, Marblehead, MA (US)

(73) Assignee: Tufts Medical Center, Inc., Boston, MA (US)

(21) Appl. No.: 13/636,413

(22) PCT Filed: Mar. 22, 2011
(86) PCT No.: PCT/US11/29297

§ 371 Date: Oct. 18, 2012

Related U.S. Application Data
(60) Provisional application No. 61/316,123, filed on Mar. 22, 2010.

(51) Int. Cl. A61B 1/06; A61M 16/04

(52) U.S. Cl. 600/120; 128/200.26

(57) ABSTRACT

A fiberoptic intubating device is provided which permits visualization of the vocal cords and automatic deployment of an endotracheal tube into the trachea upon visualization. The device includes a housing, a handle extending from the housing, and an extendable and retractable stylet extending from the distal end generally in parallel with the longitudinal axis. The device also includes a support member disposed on the housing that is configured to support the endotracheal tube with respect to the housing and to be selectively movable in the longitudinal direction relative to the housing. The device is configured to automatically move the stylet relative to the housing upon actuation of a trigger. Once the stylet is positioned relative to the vocal cords, the device is configured to deploy the endotracheal tube into the trachea upon further actuation of the trigger.

### CROSS REFERENCE TO RELATED APPLICATIONS

The present invention claims priority based on U.S. provisional patent application No. 61/316,123, filed on Mar. 22, 2010. The subject matter of this priority document is incorporated by reference herein.

### BACKGROUND OF THE INVENTION

Tracheal intubation refers to the placement of a flexible tube (an endotracheal tube) into the trachea of the body to protect the patient's airway and provide a means of mechanical ventilation. The most common tracheal intubation is orotracheal intubation where, with the assistance of an intubating device, the endotracheal tube is passed through the mouth, larynx, and vocal cords, into the trachea. However, proper intubation is difficult to achieve due to the complex anatomical arrangement in which the pharynx splits anteriorly into the trachea and posteriorly into the esophagus, and due to the fact that the endotracheal tube tends to travel posteriorly toward the esophagus during insertion. In addition, under certain circumstances, such as traumatic injury to the cervical spine, movement of the patient or the patients head, neck or lower jaw during intubation is contraindicated. In other circumstances the neck may not be able to be manipulated at all due to patient conditions such as rheumatoid arthritis or ankylosing spondylitis. In addition, patients presenting with preexisting orotracheal abnormalities can make visualization of the key anatomical structures difficult or impossible, resulting in challenges to achieving tracheal intubation.

There are many types of intubating devices which are meant to address these challenges and assist in proper placement of an endotracheal tube in the trachea, including laryngoscopes, laryngeal mask airways, stylets, and bronchoscopes. However, many of these devices have shortcomings related to obtaining adequate visualization of the anatomical structures during intubation, and accuracy and ease of flexibility in both positioning the device within the oral cavity and placement of the endotracheal tube within the trachea.

### SUMMARY

In some aspects, an intubating device includes a housing including a proximal end, a distal end opposed to the proximal end, and a longitudinal axis extending between the proximal and distal ends. The intubating device includes a handle extending from the housing, a stylet extending from the distal end generally in parallel with the longitudinal axis, and a support member disposed on the housing, the support member configured to support an endotracheal tube with respect to the housing, and to be selectively movable in the longitudinal direction relative to the housing. In addition, the intubating device includes a support member driver assembly disposed on an outer surface of the housing; and a trigger mounted on the handle and being operably connected to the support member driver assembly. The support member driver assembly is configured to drive the support member in a longitudinal direction of the housing upon actuation of the trigger.

The intubating device may include one or more of the following features: The support member driver assembly includes a worm gear disposed on the outer surface of the housing. The support member is a hollow tubular member that includes a sleeve portion having an inner diameter dimensioned to receive the distal end of the housing, and a connector portion extending from the sleeve portion, the connector portion having an outer diameter dimensioned to be press fit within an end of an endotracheal tube. In addition, a rack is formed on an inner surface of the sleeve portion, the rack configured to engage and be driven by the worm gear of the support member driver assembly relative to the housing. The support member further includes an endotracheal tube disposed on the connector portion so as to be coaxial with the stylet, and the support member driver assembly is configured to advance the support member together with the endotracheal tube along the stylet in a direction away from the handle portion upon actuation of an actuator disposed on the handle. In, addition, the support member driver assembly is configured to automatically release the endotracheal tube from the connector portion upon movement of the support member in a direction toward the handle portion. The housing includes a passageway extending between the proximal and distal ends. The stylet includes a stylet proximal end disposed in the passageway and fixed to a passageway surface at the proximal end of the housing, a stylet mid portion disposed at least partially in the passageway and extending through an opening in the distal end of the housing, and a stylet distal end disposed externally of the housing.

The intubating device may further include one or more of the following features: The intubating device further includes a stylet driver assembly disposed in the passageway and configured to position the stylet relative to the housing along the longitudinal axis. The intubating device further includes a stylet driver assembly disposed in the handle and configured to automatically drive the stylet in a longitudinal direction of the handle upon actuation of the trigger. The stylet driver assembly includes wheels arranged to abut opposed sides of the stylet, the wheels being configured to fix the stylet relative to the housing in a first operating mode, and configured to move the stylet relative to the housing along the longitudinal axis in a second operating mode. The stylet includes a stylet proximal end connected to the housing, a stylet distal end opposed to the stylet proximal end, and an optical fiber member extending from the stylet proximal end to the stylet distal end, and the stylet distal end is selectively movable in the longitudinal direction relative to the housing. The stylet includes a flexible optical fiber member extending from the stylet proximal end to the stylet distal end. The stylet includes a stylet proximal end connected to the housing, and a stylet distal end opposed to the stylet proximal end, wherein the stylet distal end is configured to be selectively angled relative to the longitudinal direction, the direction of the angle selected from directions over a 360 degree range. The stylet distal end is operably connected to an actuator mounted on the handle, the actuator configured to permit selection of the angle and angle direction. The stylet further comprises control wires extending between the stylet distal end and the handle, and the stylet distal end is positioned by manipulation of selected ones of the control wires to achieve a desired flexion angle and flexion direction of the stylet distal end. The stylet further comprises a single control wire extending between the stylet distal end and the handle, and the stylet distal end is positioned by manipulation of the single control wire to achieve a desired flexion angle relative to the longitudinal axis, and rotation of the stylet about the longitudinal axis relative to handle to achieve desired flexion direction of the stylet distal end.

Advantages of the fiber optic intubating device include the ability to easily and accurately position the leading end of the fiber optic stylet. In particular, the stylet tip has a 360 degree range of motion, and in some embodiments is actuated and controlled by a joystick mounted on the device handle. Use of a joystick, as well as placement of the joystick on the handle, allows easy and accurate positioning of the stylet tip using a single finger.

Moreover, the ability to position the stylet tip over this range of positions allows viewing of the anatomical structures, as well as easy and accurate positioning of the intubating device, often without requiring movement of the patient head and neck. Visualization of anatomical structures such the vocal cords is achieved via fiber optics provided in the stylet, and images obtained from the fiber optics may be viewed on a handle mounted LCD screen.

The fiber optic intubating device also provides a stylet tip which can be longitudinally advanced and retracted relative to the device handle through actuation of a trigger mounted on the device handle. In use, while viewing the vocal cords, the stylet tip can be advanced relative to the handle to a position between and then beyond the vocal cords into the trachea. This ability avoids the need to manually reposition the whole intubating device to achieve insertion of the stylet into the trachea, a situation in which the view of the airway can be lost during the repositioning effort. Instead, the device position, along with the view of the airway, is maintained, permitting ease of insertion of the stylet tip through the vocal cords and into the trachea.

The fiber optic intubating device further includes the ability to mechanically advance the endotracheal tube along the stylet into the trachea. Advancement of the endotracheal tube along the stylet is achieved through a further actuation of the trigger. Release of the trigger results in release of the endotracheal tube from the fiber optic intubating device. Thus, the intubating device mechanically deploys the endotracheal tube into the trachea while eliminating the need for the operator to remove a hand from the device in order to manually advance the endotracheal tube along the stylet and into the trachea, while attempting to maintain a steady position of the device with the other hand.

### BRIEF DESCRIPTION OF THE DRAWINGS

FIG. 1 is a perspective view of a fiber optic intubation device.

FIG. 2 is a partial side sectional view of the fiber optic device of FIG. 1 shown without the support member, and showing the stylet in a retracted configuration.

FIG. 3 is the side sectional view of FIG. 2 showing the stylet in an extended configuration.

FIG. 4 is a side sectional view of the fiber optic device of FIG. 1 shown with the support member and endotracheal tube assembled thereon.

FIG. 5 is an exploded side detail view of the fiber optic device of FIG. 1.

FIG. 6 is a sectional view across line 6-6 of FIG. 3.

FIG. 7 is an enlarged sectional view of the distal end of the stylet.

FIG. 8 is an enlarged side view of the distal end of the stylet.

FIG. 9A is a top sectional view of the housing.

FIG. 9B is a side sectional view of the distal end of the housing.

FIG. 9C is a sectional view across line 9C-9C of FIG. 9B.

FIG. 10 is a top view of the housing shown without the support member.

FIG. 11 is a schematic diagram of the device control system.

### DETAILED DESCRIPTION

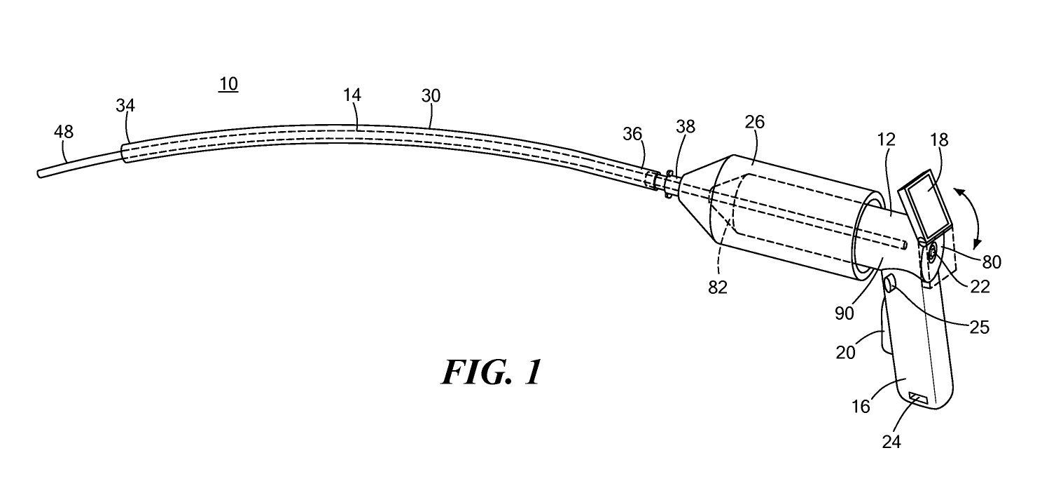

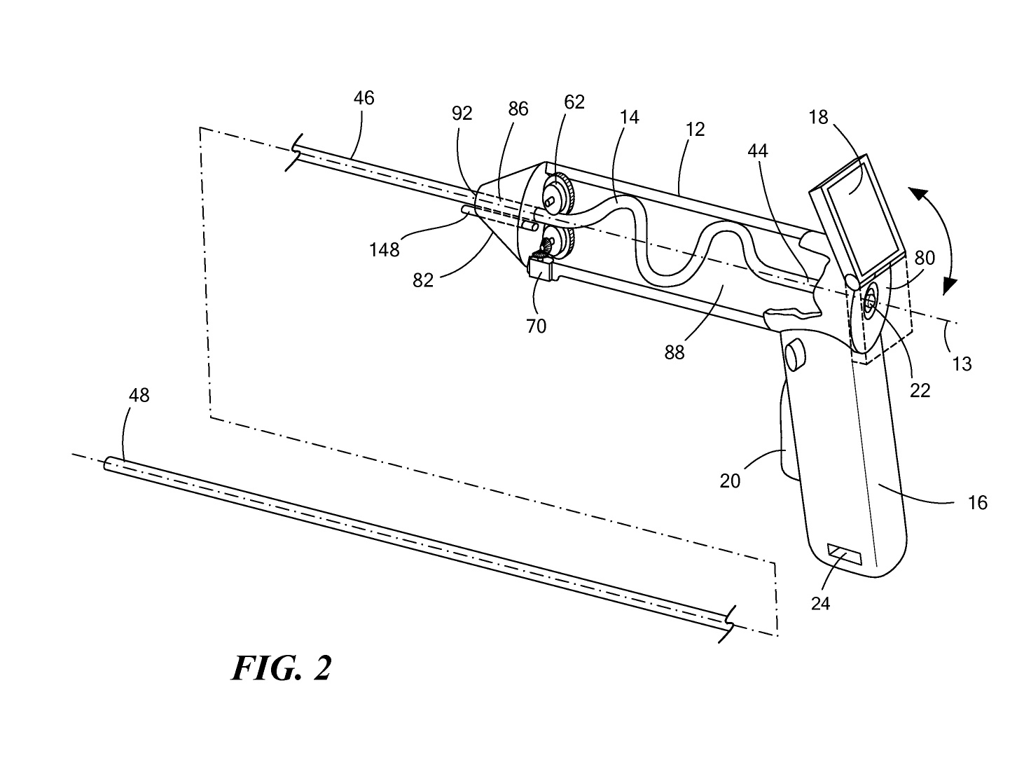

Referring now to FIGS. 1 and 2, the fiber optic intubation device 10 includes a housing 12, a handle 16 fixed to a proximal end 80 the housing 12, and a stylet 14 extending from a distal end 82 of the housing 12. The fiber optic intubation device 10 includes a support member 26 mounted on the outer surface 90 of the housing 12 for supporting and positioning an endotracheal tube 30 relative to the housing 12. The handle 16 includes a trigger 20 for actuating translational movement of the stylet 14 and/or the support member 26 relative to the housing 12, and a joystick 22 for controlling angular movement of the stylet distal end 48, as discussed further below.

An electronic display screen 18 is mounted on the proximal end 80 of the housing 12 for viewing images obtained from the distal end 48 of the stylet 14. For example, the display screen may be a liquid crystal display (LCD). The display screen 18 may be pivotally attached to the housing 12 at an edge thereof, whereby the display screen is rotatable between a first, closed position (shown in dashed lines) and a second, open position (shown in solid lines). In the closed position, the viewing surface of the display screen 18 faces toward the housing 12, providing a compact device 10 profile that is convenient for transport and storage. In the open position (shown in solid lines), the viewing surface of the display screen 18 faces away from the housing, and is substantially disposed above the housing 12. In this position, the viewing surface is easily viewed by an operator of the device 10.

The housing 12 is an elongate, generally cylindrical body which defines a longitudinal axis 13. The housing 12 includes cavity 88 adjacent to the housing proximal end 80, and a passageway 86 extending between cavity 88 and an opening 92 formed in the housing distal end 82. A portion of the stylet 14 resides within the cavity 88 and passageway 86, and then extends outward through the corresponding opening 92. A proximal end 44 of the stylet 14 is fixed to the interior surface of the cavity 88 at the proximal end 80 of the housing 12, and is electrically connected to the electronic display screen 18, a light source 28, an output port (not shown) and/or other peripheral or ancillary devices in a conventional manner.

The handle 16 extends from the proximal end 80 of the housing 12 in a direction transverse to the longitudinal axis 13, such that the overall configuration of the housing 12 and handle 16 resembles a pistol, the handle 16 providing a “pistol-grip” for holding and operating the fiber optic intubation device 10. The trigger 20 is provided on one side of the handle 16 at a location which underlies the housing 12, allowing convenient access by the index finger of an operator. The joystick 22 is positioned on the opposed side of the handle 16 relative to the trigger 20, on a rearward-facing side surface of the handle 16, allowing convenient access by the thumb of an operator. In the illustrated embodiment, the joystick 22 is covered by the display screen 18 when the display screen 18 is in the closed position. The handle 16 is also used to house a power supply 27, the light source 28, a data communication port 24 as a USB port, and control electronics 76 (FIG. 11).

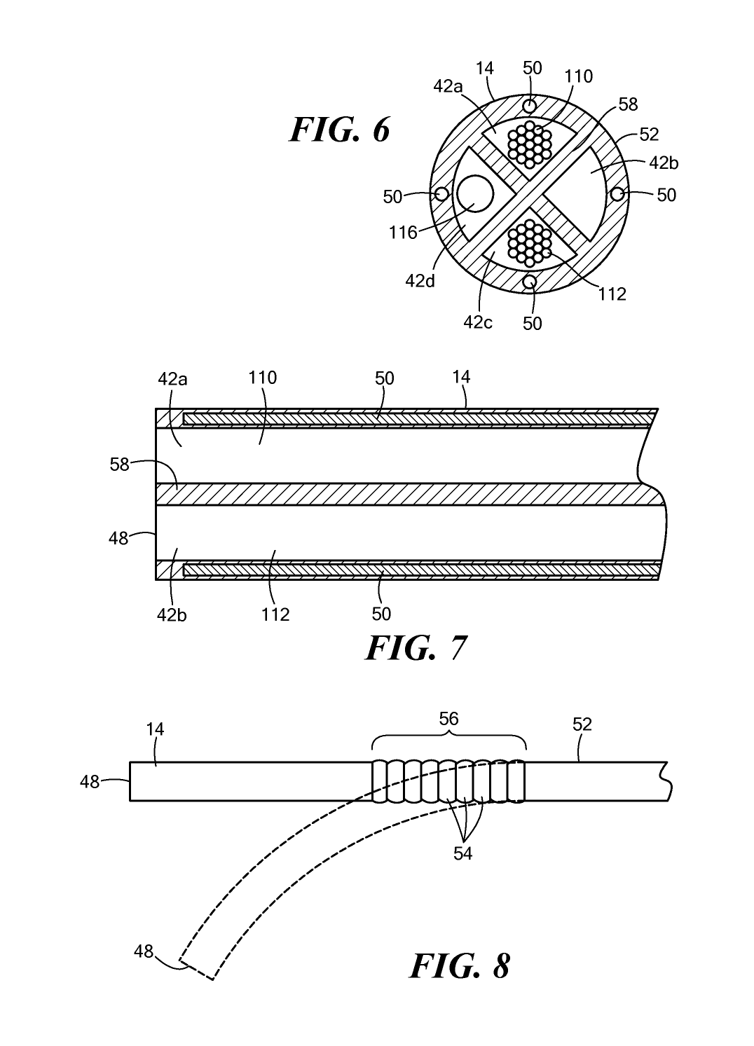

Referring to FIGS. 6-8, the stylet 14 is an elongated cylindrical tube. The outer surface 52 of the stylet 14 is flexible, and the interior space of the stylet 14 is subdivided into plural wedged-shaped channels 42a, 42b, 42c, 42d by a relatively rigid core member 58. Some regions of the stylet 14, for example a region near the distal end 48 of the stylet 14, may include links 54 to provide a localized region 56 of enhanced flexibility. In some embodiments (not shown), the flexible region 56 extends to the distal end 48. In other embodiments, the stylet 14 is formed of flexible metal tubing referred to as gooseneck tubing. The stylet 14 is sufficiently flexible to form loose coils or folds within the cavity 88 of the housing 12.

In the stylet 14, some of the channels 42 are used to house imaging devices. For example, in some embodiments, flexible plastic viewing optical fibers 110 and illuminating optical fibers 112 are disposed in opposed channels 42a, 42c. In other embodiments, an ultrasound probe 116 may be disposed in a channel 42d, in addition to or as an alternative to the optical fibers 110, 112. Images obtained from the optical fibers 110 and/or ultrasound probe 116 may be viewed on the display screen 18, or may be output via the communications port 24 for viewing on a remote display (not shown) or storage. A switch 25 may be provided on the handle 16 to permit selection of image type to be viewed. Other channels 42b are open to permit delivery of suction, ventilation, or medicines therethrough. In addition, one or more control wires 50 extend proximally from the stylet distal end 48, and are connected to a tip angle drive assembly 100. Although the stylet 14 is illustrated here as including four channels 42, a greater or fewer number of channels may be provided.

Referring to FIG. 11, the tip angle drive assembly 100 is used to control the angular position of the distal end 48 of the stylet 14. The tip angle drive assembly 100 includes one or more rotatable spools 102, and a drive motor 74 that drives the spools 102 to rotate about a respective spool axis (not shown). Each control wire 50 is fixed to a respective spool 102, and when the drive motor is actuated via the joystick 22 and associated control electronics 76, the spool 102 rotates, winding the guide wire 50 about the circumference of the spool 102. As a result, tension is applied to one or more guide wires 50. Based on joystick input, the controller 76 determines which of the guide wires 50 are actuated and the respective amount tension applied to the actuated wires 50, whereby the distal end 48 of the stylet is deflected relative to the longitudinal axis 13 in a controlled manner. Thus, the stylet distal end 48 is configured to be selectively angled relative to the longitudinal axis 13, the direction of the angle selected from directions over a 360 degree range. Moreover, by applying sufficient tension to each of the control wires 50, the position of the distal end 48 can be fixed.

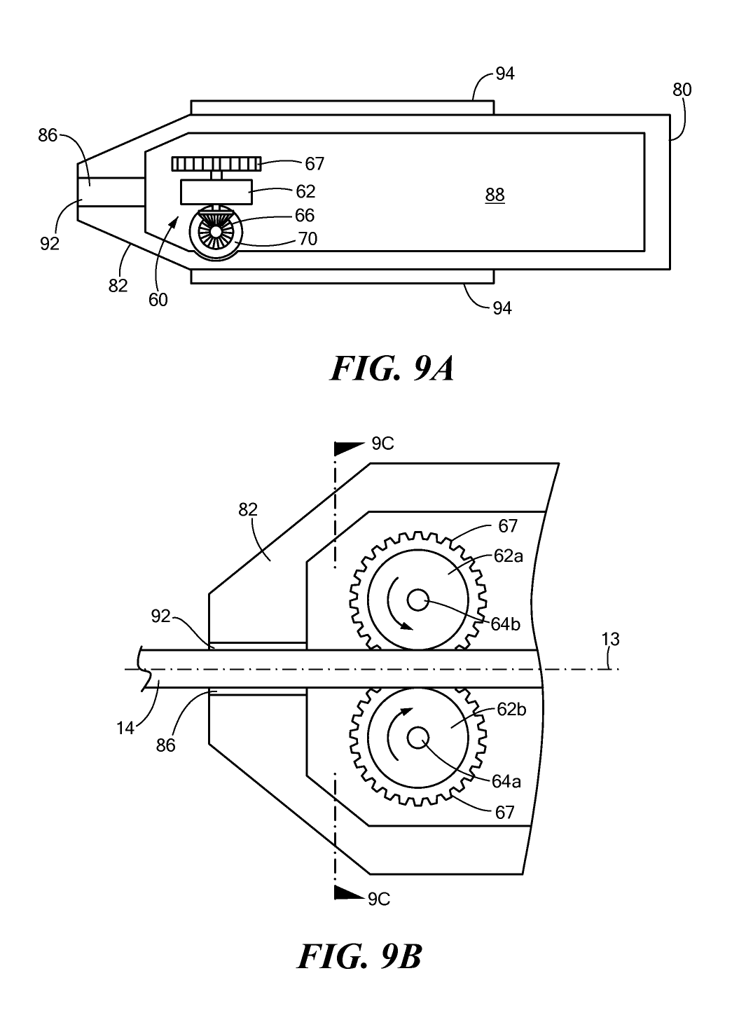

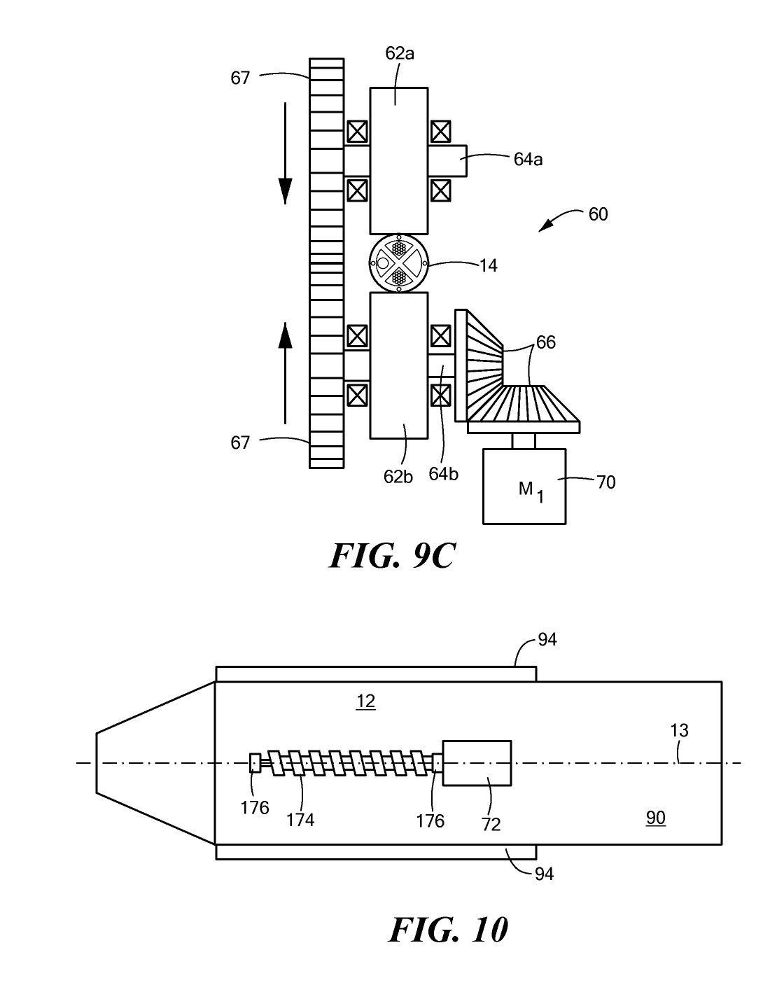

Referring to FIGS. 9A-9C, a stylet displacement drive assembly 60 is used to control the position of the distal end 48 of the stylet 14 along the direction of the longitudinal axis 13. The stylet displacement drive assembly 60 is disposed within cavity 88 adjacent to the passageway 86. The stylet displacement drive assembly 60 includes a pair of driving wheels 62a, 62b arranged to rotate in the same plane, and to be sufficiently closely spaced so that the stylet 14, passing between the respective wheel edges, is simultaneously tangent to both wheels 62a, 62b of the pair. In addition, the wheels 62a, 62b are arranged within the cavity 88 so that the respective rotational axes 64a, 64b of the wheels 62a, 62b are transverse to the longitudinal axis 13 and disposed equidistantly from, and on opposed sides of the longitudinal axis 13. In particular, the stylet 14 is in physical contact with each wheel 62a, 62b, and in some cases the spacing between the wheels 62a, 62b may be such that the stylet 14 is slightly compressed between the wheels 62a, 62b. In some embodiments, the wheels 62a, 62b are provided with a durable coating, such as a soft plastic or cork, to enable the wheels to grip the outer surface of the stylet 14 without damaging the outer surface of the stylet 14. The stylet driving assembly 60 also includes gearing 66, 67 and a drive motor 70 for driving the wheels 62a, 62b in opposed directions. The motor drive 70 is reversible, and is electrically connected to the trigger 20, and actuation of the trigger causes the motor 70 to drive the wheels 62a, 62b.

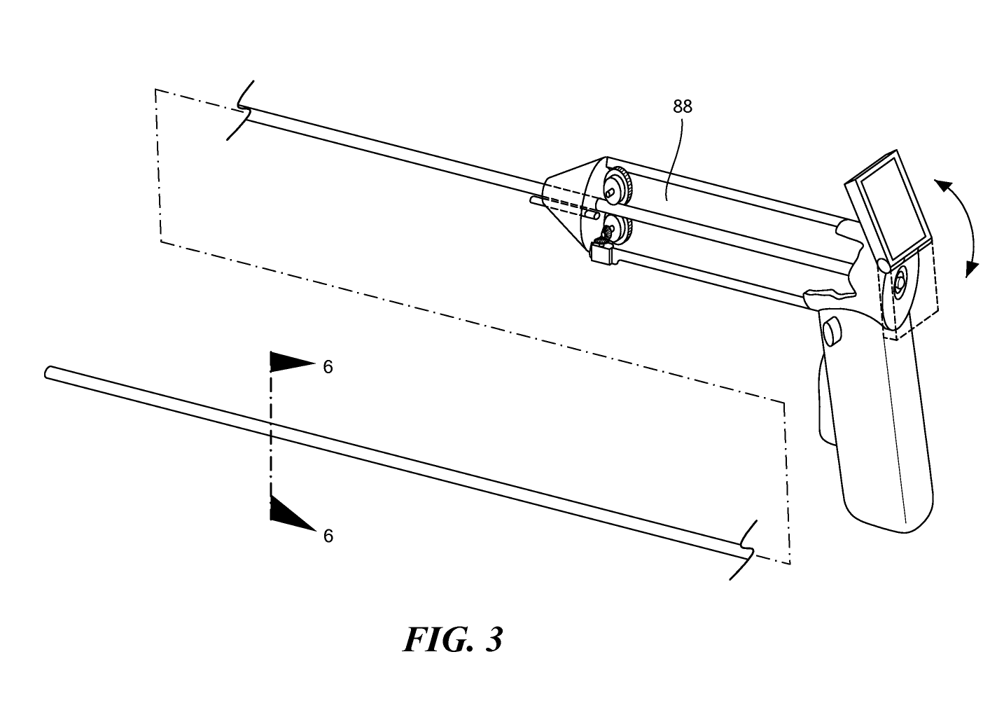

By this arrangement, the stylet 14 extends between the wheels 62a, 62b along the direction of the longitudinal axis 13, and is driven longitudinally by the wheels 62a, 62b. When the motor 70 is driven in a first direction, the wheels 62a, 62b draw the stylet into the cavity 88 of the housing 12. As the stylet 14 is drawn into the housing 12, the longitudinal distance of the distal end 48 of the stylet from the distal end 82 of the housing 12 is reduced (FIG. 2). When the motor 70 is driven in a second direction, the wheels 62a, 62b push the stylet out of the passageway 86 through the opening 92 in the distal end of the housing 12. As the stylet 14 is pushed out of the housing 12, the longitudinal distance of the distal end 48 of the stylet from the distal end 82 of the housing 12 is increased (FIG. 3). Since the proximal end 44 of the stylet 14 is fixed to the housing 12, the stylet 14 is prevented from completely exiting the housing 12, and the extent of longitudinal displacement of the stylet distal end 48 is determined by the overall length of the stylet 14. When the motor 70 is idle, the stylet 14 is prevented from longitudinal movement.

Referring again to FIG. 1, the endotracheal tube 30 is an oral, un-cuffed, single-lumen plastic tube pre-formed to curve along its longitudinal axis. The leading (insertion) end 34 of the endotracheal tube 30 is tapered, and the trailing end 36 includes a connector 38 to permit connection of the endotracheal tube 30 to an air supply source such as an ambu bag or ventilation device (not shown). Here, the connector 38 is used to secure the endotracheal tube 30 to the support member 26 of the intubation device 10, as discussed further below.

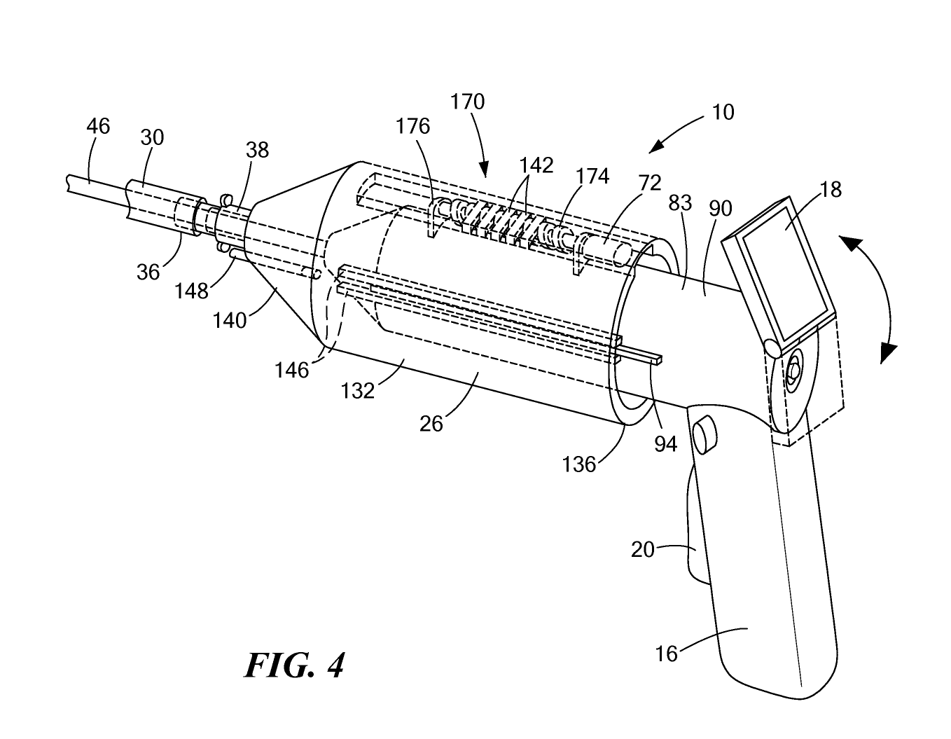

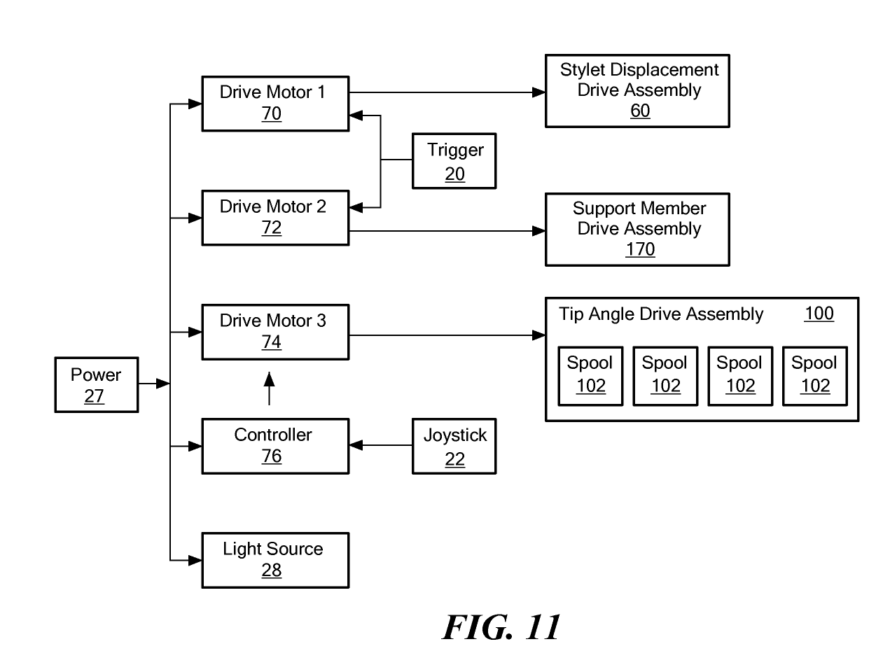

Referring also to FIGS. 4-5 and 10, the support member 26 is disposed on the distal end 82 of the housing 12 on the outer surface 90 thereof. As discussed above, the support member 26 serves to support an endotracheal tube 30 so that it detachably connects to and protrudes from the distal end 82 of the housing 12. The support member 26 is connected to the housing through the support member driver assembly 170. That is, the support member driver assembly 170 supports the support member 26 relative to the housing 12, and also permits the support member 26 to be selectively moved relative to the housing 12 upon actuation of the trigger 20. More specifically, the support member driver assembly 170 is configured to drive the support member 26 in a longitudinal direction of the housing 12 upon actuation of the trigger 20.

The support member driver assembly 170 includes a worm gear 174 disposed on the outer surface 90 of the housing 12 and aligned in parallel with the longitudinal axis 13. Opposed ends of the worm gear 174 are supported by bearings 176, and one end of the worm gear 174 is connected to a drive shaft of a drive motor 72. The drive motor 72 is reversible, and is actuated through operation of the trigger 20.

The support member 26 includes an elongate cylindrical sleeve 132 having a proximal end 136 having an inner diameter that is sufficient to enclose both the mid portion 83 of the housing 12 and the worm gear 174 disposed thereon. The distal end 138 of the sleeve 132 is of much smaller diameter than the proximal end 136, and is dimensioned to receive the connector 38 of the endotracheal tube 30 on an outer surface thereof. In some embodiments, the connector 38 is press fit on the proximal end 136 of the sleeve 132. The sleeve 132 includes a tapered portion 140 which joins the proximal and distal ends 136, 138.

An inner surface 134a of the proximal end 136 of the sleeve 132 includes a series of uniformly spaced protrusions 142 arranged to form a rack 144. The rack 144 is positioned on an upper aspect of the inner surface 134a so as to engage with and be driven by the worm gear 174 of the support member driver assembly 170. In addition, the inner surface 134a of the sleeve 132 includes guide members 146. The guide members 146 are a pair of parallel, closely spaced, longitudinally extending protrusions positioned on opposed side aspects of the inner surface 134a. The guide members 146 are dimensioned and positioned to receive the longitudinally-extending housing side rails 94. Thus, as the support member 26 is moved by the support member driver assembly 170 longitudinally relative to the housing 12, the side rails 94 slide along between, and are supported by, the pair of guide members 146. Here, interaction of the side rails 94 and guide member 146 ensures that the support member 26 moves in a longitudinal direction relative to the housing 12 without rotation of the support member 26 relative to the housing 12.

In use, an endotracheal tube 30 is disposed on the distal end 138 of the support member 26 so as to surround and be coaxial with the stylet 14. The support member driver assembly 170, which supports the support member 26 on the outer surface 90 of the housing 12, is configured to advance the support member 26 together with the endotracheal tube 30 along the stylet 14 in a direction away from the housing 12 upon actuation the trigger 20 disposed on the handle 16. In particular, the worm gear 174, which is engaged with the rack 144 on the interior surface 134a of the support member 26, rotates in a first direction upon trigger 20 actuation, driving the support member 26 distally (e.g. away from the handle 16) along the longitudinal axis 13.

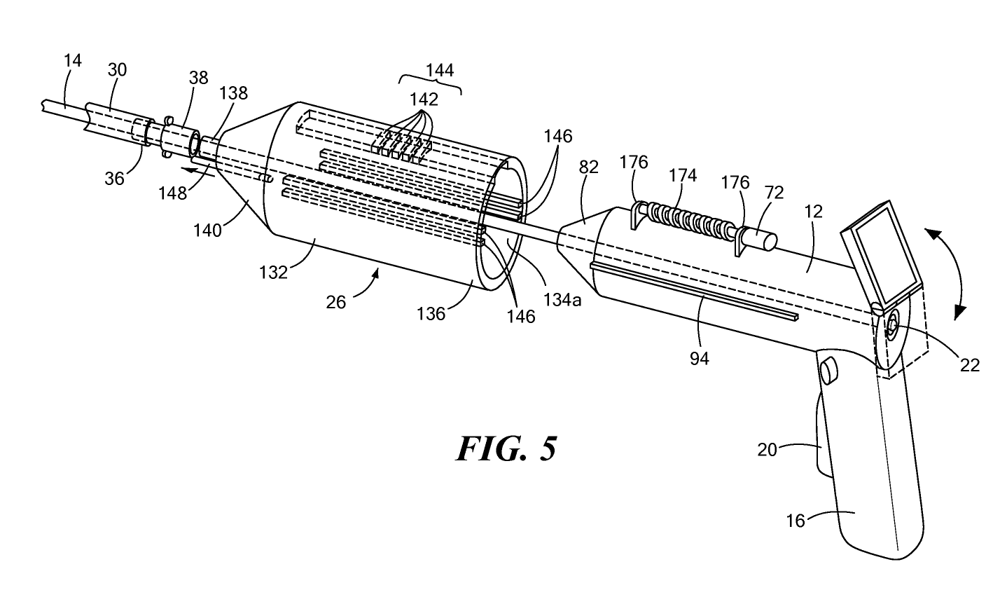

In some embodiments, release of the trigger 20 signals the motor 72 to rotate the worm gear 174 in the reverse direction, whereby the support member driver assembly 170 drives the support member 26 in a proximal direction along the longitudinal axis 13. In some embodiments, the endotracheal tube 30 is automatically released from the distal end 138 of the support member 26 upon movement of the support member 26 in a proximal direction (e.g., toward the handle 16). This is achieved, for example, by providing a deployment arm 148 which abuts the connector 38 and remains in the extended position while the support member 26 moves distally (FIG. 5). As a result, the deployment arm 148 prevents the endotracheal tube 30 from moving distally along with the support member 26, whereby the connector 38 is disengaged from the distal end 138 of the support member 26.

Operation of the fiber optic intubation device will now be described. In use, an endotracheal tube 30 is disposed on the distal end 138 of the support member 26 so as to surround and be coaxial with the stylet 14. Initially, the distal end 48 of the stylet is positioned so as to protrude distally with respect to the distal end 34. The fiber optic intubation device 10 is then inserted into the oral cavity by passing the distal end 48 of the stylet through the mouth into the larynx. Images of the vocal cords are obtained using the fiber optics 110, 112 and are viewed using the LCD 18. By referencing the images of the vocal cords, the operator operates the joystick 22 to position the distal end 48 of the stylet 14 relative to the vocal cords, and then actuates the trigger 20 to advance the distal end 48 of the stylet 14 through the vocal cords. Once the stylet 14 has passed between the vocal cords, the operator further actuates the trigger 20 to advance the support member 26, and thus the endotracheal tube 30 mounted thereon, along the stylet 14 and into the trachea. Release of the trigger 20 permits retraction of the support member 26 while leaving the endotracheal tube 30 in place in the trachea.

Use of the fiber optic intubating device 10 thus permits automatic mechanical deployment of the endotracheal tube 30 into the trachea. As discussed above, this is a great improvement over some conventional devices in which an intubating device is manually positioned until the vocal cords are in view, and then manually re-positioned in order to pass the stylet through the vocal cords. Importantly, use of the fiber optic intubating device 10 advantageously avoids the need for the operator to remove a hand from the device in order to manually advance the endotracheal tube along the stylet and into the trachea, while attempting to maintain a steady position of the device with the other hand. Instead, both the operator's hands may be maintained on the handle/housing to maintain the desired device position and achieve easy and accurate deployment of the endotracheal tube 30 into the trachea, and/or assist in mouth opening or tongue displacement.

A selected illustrative embodiment of the invention is described above in some detail. It should be understood that only structures considered necessary for clarifying the present invention have been described herein. Other conventional structures, and those of ancillary and auxiliary components of the system, are assumed to be known and understood by those skilled in the art.

Moreover, while an illustrative embodiment of the present invention has been described above, the present invention is not limited to the embodiment described above. For example, in the embodiment described above, the stylet 14 includes control wires 50 that extend between the stylet distal end 48 and the housing 12, and the stylet distal end 48 is positioned by manipulation of selected ones of the control wires 50 to achieve a desired flexion angle and flexion direction of the stylet distal end 48. In some embodiments, however, the stylet 14 instead comprises a single control wire 50 extending between the stylet distal end 48 and the housing 12, and the stylet distal end 48 is positioned by manipulation of the single control wire 50 to achieve a desired flexion angle relative to the longitudinal axis 13, followed by (or concurrent with) rotation of the stylet 14 about the longitudinal axis 13 relative to housing 12 to achieve desired flexion direction of the stylet distal end 48.

In another example, in the embodiment described above, when the mid portion 46 and proximal end 44 stylet 14 is retracted into the housing 12, the stylet 14 is sufficiently flexible to form loose coils or folds within the wide portion 88 of the passageway 84. In some embodiments, however, the mid portion 46 of the stylet 14 may instead be taken up by a spring-loaded reel (not shown) disposed in the cavity 88 of the housing 12.

In another example, in the embodiment described above, the worm gear 174 of the support member driver assembly 170 is disposed on an outer surface of the housing 12. In some embodiments, however, the worm gear 174 may instead be substantially received in a groove (not shown) formed in the surface of the housing 12, whereby only the worm gear teeth protrude outward from the housing surface 90 in order to engage the rack 144. Such a configuration results in a more compact intubation device 10.

In another example, in the embodiment described above, a single trigger 20 is provided which is depressed to a first position to operate the stylet displacement drive assembly 60, and is further depressed to a second position to operate the support member drive assembly 170. In some embodiments, however, each drive assembly 60, 170 may be provided with a dedicated trigger. In some embodiments, a switch (not shown) may be provided on the handle 16 to permit selection between rotation directions of one or more of the drive motors 70, 72.

In another example, the stylet 14 may be provided having sufficient length to permit advancement of the distal end 48 into the bronchi of the lungs. As a result, imaging of the bronchi and lung tissue, for example by fiber optic or ultra sound, can be easily obtained.

In still another example, the fiber optic intubating device 10 is described for deploying an oral, un-cuffed, single-lumen endotracheal tube 30. It is well within the scope of the invention to use the device 10 with other types of endotracheal tubes. For example, the fiber optic intubating device 10 can be used with endotracheal tubes having cuffs and/or multiple lumens, and having tapered or straight leading ends.

In still another example, one or more miniature cameras (not shown) are disposed on the distal end 48 of the stylet 14. The cameras may be provided in addition to, or as an alternative to, the optical fibers 110, 112 and/or ultrasound probe 116. Image data obtained by the cameras can be transmitted by wires or wirelessly to the display screen 18. In some embodiments, the miniature cameras on the distal end 48 and the display screen 18 will support 3-D technology.

Thus, various design alterations may be carried out without departing from the present invention as set forth in the claims.

What is claimed is,:

1. An intubating device comprising
a housing including a proximal end, a distal end opposed to the proximal end, and a longitudinal axis extending between the proximal and distal ends;
a handle extending from the housing;
a stylet extending from the distal end generally in parallel with the longitudinal axis;
a support member disposed on the housing, the support member configured to support an endotracheal tube with respect to the housing, and to be selectively movable in the longitudinal direction relative to the housing; and
a support member driver assembly disposed on an outer surface of the housing; and
a trigger mounted on the handle and being operably connected to the support member driver assembly,
wherein the support member driver assembly is configured to drive the support member in a longitudinal direction of the housing upon actuation of the trigger.

2. The intubating device of claim 1 wherein
the support member driver assembly includes a worm gear disposed on the outer surface of the housing, and
the support member is a hollow tubular member that includes
a sleeve portion having an inner diameter dimensioned to receive the distal end of the housing, and
a connector portion extending from the sleeve portion, the connector portion having an outer diameter dimensioned to be press fit within an end of an endotracheal tube,
a rack formed on an inner surface of the sleeve portion, the rack configured to engage and be driven by the worm gear of the support member driver assembly relative to the housing.

3. The intubating device of claim 2 wherein the support member further comprises an endotracheal tube disposed on the connector portion so as to be coaxial with the stylet, and
the support member driver assembly is configured to
advance the support member together with the endotracheal tube along the stylet in a direction away from the handle portion upon actuation of an actuator disposed on the handle, and
automatically release the endotracheal tube from the connector portion upon movement of the support member in a direction toward the handle portion.

4. The intubating device of claim 1 wherein
the housing includes a passageway extending between the proximal and distal ends, and
the stylet includes
a stylet proximal end disposed in the passageway and fixed to a passageway surface at the proximal end of the housing,
a stylet mid portion disposed at least partially in the passageway and extending through an opening in the distal end of the housing, and
a stylet distal end disposed externally of the housing.

5. The intubating device of claim 4 further comprising a stylet driver assembly disposed in the passageway and configured to position the stylet relative to the housing along the longitudinal axis.

6. The intubating device of claim 4 further comprising a stylet driver assembly disposed in the handle and configured to automatically drive the stylet in a longitudinal direction of the handle upon actuation of the trigger.

7. The intubating device of claim 5 wherein the stylet driver assembly includes wheels arranged to abut opposed sides of the stylet, the wheels configured to fix the stylet relative to the housing in a first operating mode, and configured to move the stylet relative to the housing along the longitudinal axis in a second operating mode.

8. The intubating device of claim 1 wherein the stylet includes
a stylet proximal end connected to the housing, a stylet distal end opposed to the stylet proximal end, and a flexible optical fiber member extending from the stylet proximal end to the stylet distal end,
wherein the stylet distal end is selectively movable in the longitudinal direction relative to the housing.

9. The intubating device of claim 1 wherein the stylet includes
a stylet proximal end connected to the housing, a stylet distal end opposed to the stylet proximal end, and an optical fiber member extending from the stylet proximal end to the stylet distal end,
wherein the stylet distal end is selectively movable in the longitudinal direction relative to the housing.

10. The intubating device of claim 1 wherein the stylet includes
a stylet proximal end connected to the housing, and a stylet distal end opposed to the stylet proximal end,
wherein the stylet distal end is configured to be selectively angled relative to the longitudinal direction, the direction of the angle selected from directions over a 360 degree range.

11. The intubating device of claim 10 wherein the stylet distal end is operably connected to an actuator mounted on the handle, the actuator configured to permit selection of the angle and angle direction.

12. The intubating device of claim 10 wherein the stylet further comprises control wires extending between the stylet distal end and the handle, and the stylet distal end is positioned by manipulation of selected ones of the control wires to achieve a desired flexion angle and flexion direction of the stylet distal end.

13. The intubating device of claim 10 wherein the stylet further comprises a single control wire extending between the stylet distal end and the handle, and the stylet distal end is positioned by
manipulation of the single control wire to achieve a desired flexion angle relative to the longitudinal axis, and
rotation of the stylet about the longitudinal axis relative to handle to achieve desired flexion direction of the stylet distal end.
